(19) United States
(12) Patent Application Publication
(10) Pub. No.: US 2017/0325504 A1
(43) Pub. Date: Nov. 16, 2017

(54) DISPOSABLE ELECTRONIC CIGARETTE

(71) Applicant: HUIZHOU KIMREE TECHNOLOGY CO., LTD., Huizhou, Guangdong (CN)

(72) Inventor: Qiuming Liu, Shenzhen, Guangdong (CN)

(21) Appl. No.: 15/522,786

(22) PCT Filed: Oct. 31, 2014
(86) PCT No.: PCT/CN2014/090087

§ 371 Date: Apr. 28, 2017

(51) Int. Cl. A24F 47/00; A61M 11/04; A61M 15/06

(52) U.S. Cl. CPC A24F 47/008; A61M 15/06; A61M 11/042; A61M 2205/8206

(57) ABSTRACT

A disposable electronic cigarette comprising a sleeve, an atomizing assembly and a battery component. The atomizing assembly comprises an e-liquid cup for storing e-liquid; a cup cover connected to the e-liquid cup and covering an opening of the e-liquid cup, the cup cover is provided with e-liquid permeating holes for guiding the e-liquid out from the e-liquid cup; a heating wire assembly for atomizing the e-liquid outputted from the e-liquid permeable holes; an atomizing seat for fixing the heating wire assembly, the atomizing seat and the cup cover enclose to form an atomizing cavity for accommodating the heating wire assembly. Provided in the axial direction of the atomizing seat are conductive devices and a force applying device used for applying a force in a direction toward the battery component to the conductive devices. An elastic sealing device is provided between the atomizing assembly and the battery component.

### FIELD OF THE INVENTION

The present application relates to a technical field of an electronic cigarette, and more particularly relates to a disposable electronic cigarette.

### BACKGROUND OF THE INVENTION

A disposable electronic cigarette an electronic cigarette which has no need to be charged, no need to replace e-liquid and can be discarded after once use. Compared with a rechargeable electronic cigarette, the structure of the disposable electronic cigarette is relatively simpler and cost is lower.

In the prior art, the disposable electronic cigarette has the following defects, in an assembly process, when a soft cotton material is used to store smoke oil, the soft cotton material is not easy to be inserted into a preset position within a sleeve, and during a using process, an e-liquid storage cotton is easy to be squeeze the e-liquid out under a pressure of an external force, and the e-liquid storage cotton is prone to produce e-liquid leakage phenomenon, thus affecting a smoking effect and producing short circuit phenomenon of an electronic cigarette circuit; An electrical connection between the atomizing assembly and the battery component is unstable, thus the electronic cigarette does not work properly; An electric heating wire is in a spiral plate shape, and e-liquid contact area is too large and heat quickly spread to the e-liquid, then the e-liquid cannot be atomized or need more power, thereby affecting the user smoking experience; No airflow channel has been defined at the atomizing assembly end, thus atomized smoke cannot be discharged, thereby affecting the user's smoking effect; A microphone head is not be sealed and isolated, then the microphone head cannot receive a signal triggered and sensed by the air resulting in that the electronic cigarette cannot work.

Therefore, it is important to make improvements to the disposable electronic cigarette of the prior art.

### SUMMARY OF THE INVENTION

Technical problems to be solved in the present invention is to provide a disposable electronic cigarette of which atomizing assembly is connected stably with the battery component, so that the atomized smoke cannot enter the battery component aiming at defects in the prior art.

Technical problems to be solved in the present invention is to provide a disposable electronic cigarette comprising a sleeve, an atomizing assembly and a battery component which are sleeved within the sleeve;

The atomizing assembly comprises an e-liquid cup provided with an opening at an end of the e-liquid cup and configured for storing an e-liquid, the end of the e-liquid cup faces the battery component; at least one smoke passage is defined between the sleeve and the e-liquid cup for discharging atomized smoke outside one end of the atomizing assembly; a cup cover is connected to the e-liquid cup and covers the opening of the e-liquid cup, the cup cover is provided with e-liquid permeating holes for guiding the e-liquid out of the e-liquid cup; a heating wire assembly is provided for atomizing the e-liquid outputted from the e-liquid permeating holes; an atomizing seat is provided for fixing the heating wire assembly, an atomizing chamber for receiving the heating wire assembly is enclosed between the atomizing seat and the cup cover, the atomizing chamber is communicated with the smoke passage;

The electronic cigarette further comprises conductive devices and a force applying device, the conductive devices are defined axially along the atomizing seat, the force applying device is configured for applying a force on the conductive devices in a direction to the battery component, one ends of the conductive devices are connected to the heating wire assembly, and the other ends of the conductive devices are elastically abutted against the battery component by a force of the force applying device; an elastic sealing device for preventing the e-liquid in the atomizing assembly from leaking toward the battery component is defined between the atomizing assembly and the battery component, the elastic sealing device is elastically abutted against an inner wall of the sleeve.

In the disposable electronic cigarette provided in the present invention, an air inlet hole and an air outlet hole are defined on a side wall of the atomizing chamber, the side wall of the atomizing chamber faces the sleeve, the air inlet hole and the air outlet hole are opposite to each other, the air inlet hole is communicated with the outside of the electronic cigarette, the air outlet hole is communicated with the smoke passage.

In the disposable electronic cigarette provided in the present invention, an e-liquid collection tank for collecting leaked and condensed e-liquid is formed in one end of the atomizing chamber, the end of the atomizing chamber is opposite to the e-liquid cup.

In the disposable electronic cigarette provided in the present invention, the air inlet hole and the air outlet hole are provided on a circumferential side wall of the cup cover, a circumference of one end of the e-liquid cup extends along a direction opposite to the cup cover to form an abutting sleeve, the one end of the e-liquid cup is opposite to the cup cover, a smoke hole corresponding to the air outlet hole is provided in a circumferential side wall of the abutting sleeve;

The electronic cigarette further comprises an air intake passage, the air intake passage, the atomizing chamber and the smoke passage form an airflow passage together.

In the disposable electronic cigarette provided in the present invention, the sleeve is provided with an air intake port corresponding to the air inlet hole, outside air enters into the atomizing chamber by sequentially passing through the air intake port and the air inlet hole, so as to form the air intake passage.

In the disposable electronic cigarette provided in the present invention, a first sealing seat is defined between the cup cover and the sleeve, one side of the first sealing seat close to the air outlet hole is provided with a ventilation groove for communicating the atomizing chamber with the smoke passage, smoke flows from the atomizing chamber into the smoke passage through the ventilation groove and is discharged from the smoke passage.

In the disposable electronic cigarette provided in the present invention, the heating wire assembly comprises an electric heating wire and an e-liquid guiding wick, the e-liquid guiding wick is configured for guiding e-liquid, the electric heating wire is winding around the e-liquid guiding wick, two through holes spaced from each other are axially defined in the atomizing seat, each of the through holes is provided with one of the conductive devices, each of the conductive devices comprises a fixing sleeve and an elastic column, a first axial mounting position and a second axial mounting position are respectively and correspondingly defined in the fixing sleeve, the elastic column is elastically and movably mounted in the first axial mounting position, two ends of the electric heating wire are respectively and correspondingly inserted into two second axial mounting positions of the conductive devices.

In the disposable electronic cigarette provided in the present invention, the elastic column comprises an abutment member movably defined in the first axial mounting position and configured for being electrically connected to the battery component, and an elastic member provided in the first axial mounting position for providing an elastic force for the abutment member and resetting the abutment member, when the battery component is connected to the atomizing assembly, the battery in the battery component is electrically connected with the abutment member.

In the disposable electronic cigarette provided in the present invention, an e-liquid separator for blocking a leakage of e-liquid is provided in the cup cover, the e-liquid permeating holes are defined on the e-liquid separator, an e-liquid storage cotton is abutted against an end surface of the e-liquid separator, the end surface of the e-liquid separator is opposite to e-liquid, the e-liquid storage cotton guides the e-liquid out from the e-liquid permeating holes; a fiberglass sheet is abutted against an end surface of e-liquid storage cotton, the end surface of e-liquid storage cotton is opposite to the e-liquid separator, the electric heating wire is wound around the e-liquid guiding wick, the e-liquid guiding wick is abutted against the fiberglass sheet.

In the disposable electronic cigarette provided in the present invention, an e-liquid separator for blocking a leakage of e-liquid is provided in the cup cover, the e-liquid permeating holes are defined on the e-liquid separator, two ends of the e-liquid guiding wick are respectively inserted into the e-liquid permeating holes and extend to the e-liquid cup to directly contact with the e-liquid.

In the disposable electronic cigarette provided in the present invention, the electronic cigarette further comprises an end cap inserted into one end of the sleeve, the end of the sleeve is close to the battery component, and a second sealing seat is defined between the battery component and the end cap;

A first intake slot is defined on the end cap, a second intake slot corresponding to the first intake slot is defined on the second sealing seat, a center through hole is defined on the atomizing seat, outside air successively passes through the first intake slot, the second intake slot and the center through hole to enter the atomizing chamber.

In the disposable electronic cigarette provided in the present invention, the battery component is co-axially defined with the atomizing assembly, and the battery component and the atomizing assembly are defined within the sleeve, a first gap is defined between the atomizing assembly and the sleeve, the first gap forms the smoke passage, a second gap is defined between the battery component and the sleeve, and the second gap forms an air intake passage.

In the disposable electronic cigarette provided in the present invention, the electronic cigarette further comprises a suction nozzle cover inserted into one end of the sleeve close to the e-liquid cup and abutted against the e-liquid cup, the suction nozzle cover is provided with an opening, smoke is discharged through the opening after passing through the smoke passage.

In the disposable electronic cigarette provided in the present invention, the battery component comprises a circuit board abutted against the conductive devices, a battery, a FPC board, an airflow sensor and an airflow sensor seat, the airflow sensor seat is configured for accommodating the airflow sensor, the circuit board and the airflow sensor seat are defined at two ends of the FPC board, respectively, the battery is provided on a side of the FPC board and is located between the circuit board and the airflow sensor seat, the FPC board is configured for realizing an electrical connection between the circuit board and the airflow sensor;

A second sealing seat is sleeved on the battery and the airflow sensor seat and is defined at one end of the battery component, the elastic sealing device sleeves on the circuit board and sleeves on the other end of the battery component to enable the battery component to be inserted into one end of the sleeve with the elastic sealing device and the second sealing seat being sleeved on two ends of the battery component when assembling.

In the disposable electronic cigarette provided in the present invention, one end of the FPC board is provided with three conductive holes, the end of the FPC board is close to the airflow sensor, one of the conductive holes is in contact with one of three contact points on the airflow sensor, the conductive holes are also in electrical contact with one end of the battery, the other end of the FPC board is in electrical contact with the other end of the battery, the battery, the airflow sensor and the airflow sensor seat are embedded in a frame chamber of the FPC board and are limited by mutually pressing of limiting boards defined at two ends of the FPC board to achieve an electrical connection.

In the disposable electronic cigarette provided in the present invention, the cup cover comprises an e-liquid separator, a first connecting portion connected to one end of the e-liquid separator and inserted into the e-liquid cup, a second connecting portion connected to the other end of the e-liquid separator, an air inlet hole and an air outlet hole are oppositely provided in a circumferential side wall of the second connecting portion, and an outer peripheral wall of the e-liquid separator is provided with a placing groove for placing a sealing ring.

In the disposable electronic cigarette provided in the present invention, the atomizing seat is inserted into the second connecting portion, the atomizing seat and the second connecting portion enclose to form the atomizing chamber; the second connecting portion and the atomizing seat are abutted against the elastic sealing device, and a gap for smoke to flow through is provided between an outer side wall of the e-liquid separator and the sleeve.

In the disposable electronic cigarette provided in the present invention, an outer surface of the e-liquid cup comprises an axially extending section and a remaining surface, the section and an inner wall of the sleeve form the smoke passage, the remaining surface is attached to the inner wall of the sleeve.

In the disposable electronic cigarette provided in the present invention, the force applying device comprises a first magnet and a second magnet, the first magnet and the second magnet are defined on the atomizing seat and the battery component, respectively.

In the disposable electronic cigarette provided in the present invention, the electronic cigarette further comprises a suction nozzle cover inserted into the one end of sleeve, the one end of sleeve is close to the e-liquid cup; the force applying device is a spring, two ends of the spring are abutted against an end of the suction nozzle cover and an end of the e-liquid cup, respectively.

Applications of the disposable electronic cigarette of the present invention have following advantages, through a configuration of the e-liquid cup, the e-liquid cup can be more conveniently inserted into the sleeve directly in the assembly process of the electronic cigarette, so as to avoid the problem that for the disposable electronic cigarette of the prior art, when the soft cotton material is used to store e-liquid, the soft cotton material is not easy to be inserted into a preset position within a sleeve in an assembly process, and also to avoid the problem that during a using process, an e-liquid storage cotton is easy to squeeze the e-liquid out under a pressure of an external force, so the structure of the present invention is more convenient for an automated production, improves production efficiency, and to well prevent an e-liquid leakage during the use of the disposable as the disposable electronic cigarette is pressed;

Further, by applying the force applying device on the conductive devices in a direction to the battery component, the conductive devices can be brought into close to contact with the battery component, besides, the conductive devices are elastic devices, thereby eliminating phenomenon of an electrical contact failure; A elastic sealing device defined between the atomizing assembly and the battery component prevents the atomized smoke from entering the battery component to affect a normal operation of the battery;

Further, since the second sealing seat is sleeved on the battery and the airflow sensor seat and is located at one end of the battery component, the elastic sealing device sleeves the circuit board and is simultaneously sleeves on the other end of the battery component, so that during an assembly, the battery component having the elastic sealing device and the second sealing seat defined at two ends is inserted into one end of the sleeve, thereby facilitating the automatic production and improving the production efficiency.

Further, by providing the air intake port on the sleeve, airflow is introduced into the atomizing chamber through the air intake port to carry the smoke out from the other side of the e-liquid cup to an user mouth, and the airflow path is prevented from being bent too many times to produce phenomenon of airflow noise and suffocation; The first sealing seat is defined on the outer peripheral surface of the atomizing chamber, and a ventilation groove is defined on one side of the first sealing seat so that the smoke is discharged from the ventilation groove to prevent the atomized smoke from accumulating in the atomizing chamber to affect smoke taste and user experience; Through a small abutting area between surfaces of the electric heating wire and the fiberglass sheet to ensure a smooth e-liquid transmission, but also to prevent that the contact area between the electric heating wire and the e-liquid is too large to lose the heat fast resulting in phenomenon that the e-liquid cannot be atomized; By directing two ends of the e-liquid guiding wick into the e-liquid cup and the direct contact with the e-liquid, it is possible to avoid the phenomenon that the e-liquid provided for the electric heating wire to atomize is insufficient to produce burning cotton phenomenon; By embedding the airflow sensor seat and the airflow sensor into the second sealing seat, it is possible to prevent the airflow sensor from receiving no triggered and sensed signal due to a small air pressure difference at two ends of the airflow sensor then the electronic cigarette cannot work normally.

### BRIEF DESCRIPTION OF THE DRAWINGS

The present invention will be further described with reference to the accompanying drawings and embodiments in the following.

FIG. 1 is a schematic view of a three-dimensional structure of a disposable electronic cigarette of the present invention;

FIG. 2 is an explosion diagram of the disposable electronic cigarette of the present invention;

FIG. 3 is a front view of a first embodiment of the disposable electronic cigarette of the present invention;

FIG. 4 is an enlarged view of the B region in FIG. 3;

FIG. 5 is a schematic view of a partial structure of the disposable electronic cigarette of the present invention;

FIG. 6 is a schematic view of the three-dimensional structure of an e-liquid cup of the disposable electronic cigarette of the present invention;

FIG. 7 is a schematic view of a three-dimensional structure of a heating wire assembly according to a first embodiment of the disposable electronic cigarette of the present invention;

FIG. 8 is an explosion diagram of the atomizing assembly of the first embodiment of the disposable electronic cigarette of the present invention;

FIG. 9 is a schematic view of a three-dimensional structure of an atomizing seat according to the first embodiment of the disposable electronic cigarette of the present invention;

FIG. 10 is a front view of one conductive device of the disposable electronic cigarette of the present invention;

FIG. 11 a schematic view of a three-dimensional structure of an end cap of the disposable electronic cigarette according to the present invention;

FIG. 12 is a schematic view of the three-dimensional structure of a heating wire assembly of a second embodiment of the disposable electronic cigarette of the present invention;

FIG. 13 is a front view of a third embodiment of the disposable electronic cigarette of the present invention;

FIG. 14 is an enlarged view of the section A in FIG. 12;

FIG. 15 is an explosion view of an atomizing assembly of the third embodiment of the disposable electronic cigarette of the present invention;

FIG. 16 is a schematic view of the three-dimensional structure of an end cap and a sleeve of the disposable electronic cigarette of the present invention;

FIG. 17 is a schematic view of a three-dimensional structure of the atomizing seat according to the third embodiment of the disposable electronic cigarette of the present invention;

FIG. 18 is an explosion diagram of the battery component, the elastic sealing device and the second sealing seat of the disposable electronic cigarette of the present invention.

### DETAILED DESCRIPTION OF THE PREFERRED EMBODIMENT

To make the technical feature, objective and effect of the present application be understood more clearly, now the specific implementation of the present application is described in detail with reference to the accompanying drawings and embodiments.

As shown in FIGS. 1 to 18, the present invention provides a disposable electronic cigarette, the disposable electronic cigarette comprises a sleeve 1, an atomizing assembly 2 and a battery component 3, the atomizing assembly 2 and the battery component 3 are sleeved within the sleeve 1, the atomizing assembly 2 comprises an e-liquid cup 21 provided with an opening at an end of the e-liquid cup 21 and configured for storing an e-liquid, the end of the e-liquid cup 21 faces the battery component 3; at least one smoke passage is defined between the sleeve 1 and the e-liquid cup 21 for discharging the atomized smoke outside one end of the atomizing assembly 2; a cup cover 22 is inserted into the e-liquid cup 21 and covers the opening of the e-liquid cup 21, the cup cover is 22 provided with e-liquid permeating holes 222 for guiding the e-liquid out of the e-liquid cup 21; a heating wire assembly 23 for atomizing the e-liquid outputted from the e-liquid permeating holes 222; an atomizing seat 24 for fixing the heating wire assembly 23, an atomizing chamber for receiving the heating wire assembly 23 is enclosed by the atomizing seat 24 and the cup cover 22, the atomizing chamber is communicated with the smoke passage.

The electronic cigarette further comprises conductive devices 241 and a force applying device, the conductive devices 241 are defined axially along the atomizing seat 24, the force applying device is configured for applying a force on the conductive devices 241 in a direction to the battery component 3, one ends of the conductive devices 241 are connected to the heating wire assembly 23, and the other ends of the conductive devices 241 are elastically abutted against the battery component 3 by a force of the force applying device; an elastic sealing device 201 for preventing the e-liquid in the atomizing assembly 2 from leaking toward the battery component 3 is defined between the atomizing assembly 2 and the battery component 3, the elastic sealing device 201 is elastically abutted against an inner wall of the sleeve 1. By applying the force applying device on the conductive devices 241 in a direction to the battery component 3, the conductive devices 241 can be brought into close to contact with the battery component 3, besides, the conductive devices 241 are elastic devices, thereby eliminating phenomenon of an electrical contact failure; An elastic sealing device 201 defined between the atomizing assembly 2 and the battery component 3 prevents atomized smoke from entering the battery component 3 to affect a normal operation of the battery.

The force applying device comprises a first magnet and a second magnet (not shown in drawings), the first magnet and the second magnet are defined on the atomizing seat 24 and the battery component 3, respectively. Specifically, the first magnet is provided on an end surface of the atomizing seat 24 close to the atomizing chamber, and the second magnet is provided on an end surface of the battery component 3 close to the atomizing seat 24, the first magnet and the second magnet are attracted to each other so that the atomizing seat 24 and the battery component 3 are brought close to each other under an action of the magnet, one ends of the conductive devices 241 are kept being abutted against the battery component 3 to avoid phenomenon of poor electrical contact. In addition, even if the conductive devices 241 are disconnected from the battery component 3 due to vibration or the like, the conductive devices 241 are automatically connected to the battery component 3 due to mutual suction between the first magnet and the second magnet, thereby the conductive devices 241 are brought into elastic contact with the battery component 3.

Besides, as shown in FIG. 5, the force applying device may be a spring 6, specifically, an inner end surface of a suction nozzle cover 4 is abutted against one end of the spring 6, and the other end of the spring 6 is abutted against one end of the e-liquid cup 21, when the suction nozzle cover 4 is pressed, one ends of the conductive devices 241 fixed to the e-liquid cup 21 is kept being abutted against the battery component 3 by an elastic force of the spring 6 to avoid an occurrence of the electrical contact failure.

It is to be understood that in the present invention, the force applying device is configured for applying force on the conductive devices 241 in a direction to the battery component 3 so as to avoid the phenomenon of the poor electrical contact, but the force applying device of the present invention may be in other forms, as long as it can avoid the phenomenon of the poor electrical contact.

In addition, the atomizing chamber is enclosed by the atomizing seat 24 and the cup cover 22, an e-liquid collection tank for collecting leaked and condensed e-liquid is formed in one end of the atomizing chamber opposite to the e-liquid cup 21, leaked or condensed e-liquid is stored in the e-liquid collection tank to prevent the e-liquid from entering the battery component 3 and affect the normal operation of the battery component 3.

### A First Embodiment

As shown in FIG. 1, in the first embodiment of the present invention, the disposable electronic cigarette comprises a sleeve 1, a suction nozzle cover 4 and an end cap 5, the suction nozzle cover 4 and the end cap 5 respectively covered at two ends of the sleeve 1, the sleeve 1 is cylindrical, the suction nozzle cover 4 is configured for the user to inhale smoke, and the disposable electronic cigarette of the present invention simulates an appearance of a real cigarette so as to achieve an effect of simulating smoking of the real cigarette.

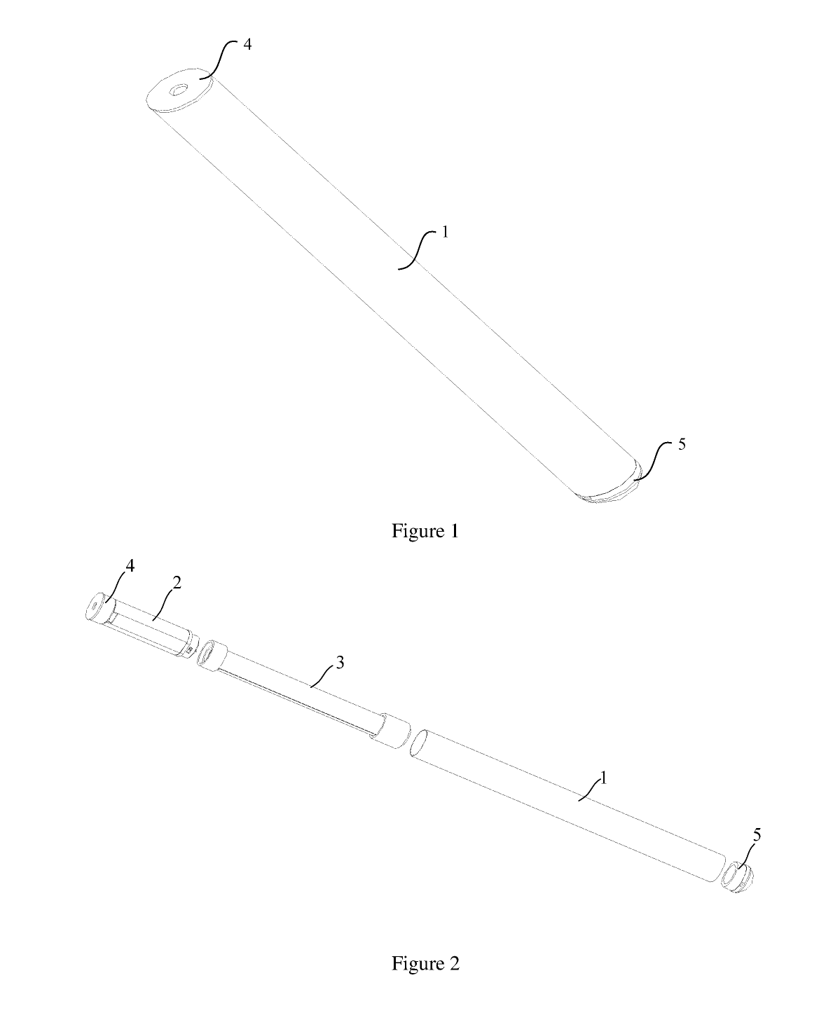

Specifically, as shown in FIG. 2, the disposable electronic cigarette further comprises an atomizing assembly 2 and a battery component 3 housed in the sleeve 1, and the atomizing module 2 and the battery component 3 are co-axially defined within the sleeve 1. The atomizing assembly 2 is defined at one end of the sleeve 1 close to the suction nozzle cover 4, and the battery component 3 is defined at one end of the sleeve 1 close to the end cap 5. During assembly, the battery component 3 and the atomizing assembly 2 can be sequentially inserted into the sleeve 1, thereby facilitating an automated production.

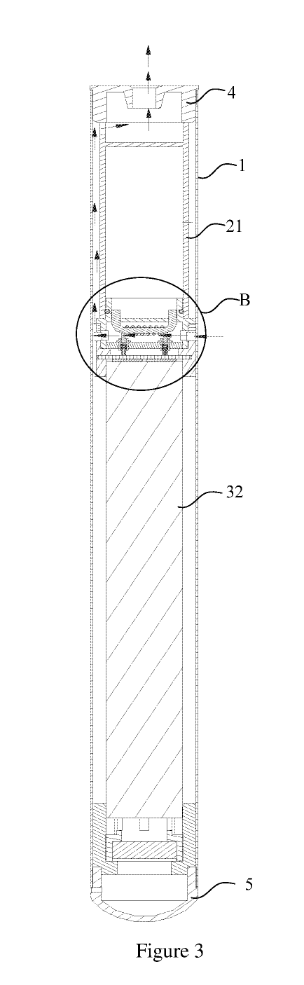

As shown in FIG. 3, in the first embodiment of the present invention, the disposable electronic cigarette comprises the sleeve 1, the suction nozzle cover 4, the end cap 5, and an e-liquid cup 21 and a battery 32, the suction nozzle cover 4 and the end cap 5 are provided at two ends of the sleeve 1, and the e-liquid cup 21 and the battery 32 are co-axially defined in the sleeve 1, and the e-liquid cup 21 is defined at one end of the sleeve 1 close to the suction nozzle cover 4, and the battery 32 is defined at one end of the sleeve 1 close to the end cap 5. Airflow enters the atomizing chamber from one side of the sleeve 1 located at a part between the e-liquid cup 21 and the battery 32 and carries smoke from the other side of the sleeve 1 to the user's mouth to avoid the airflow channel bending too many times to result in phenomenon of airflow noise and suffocation.

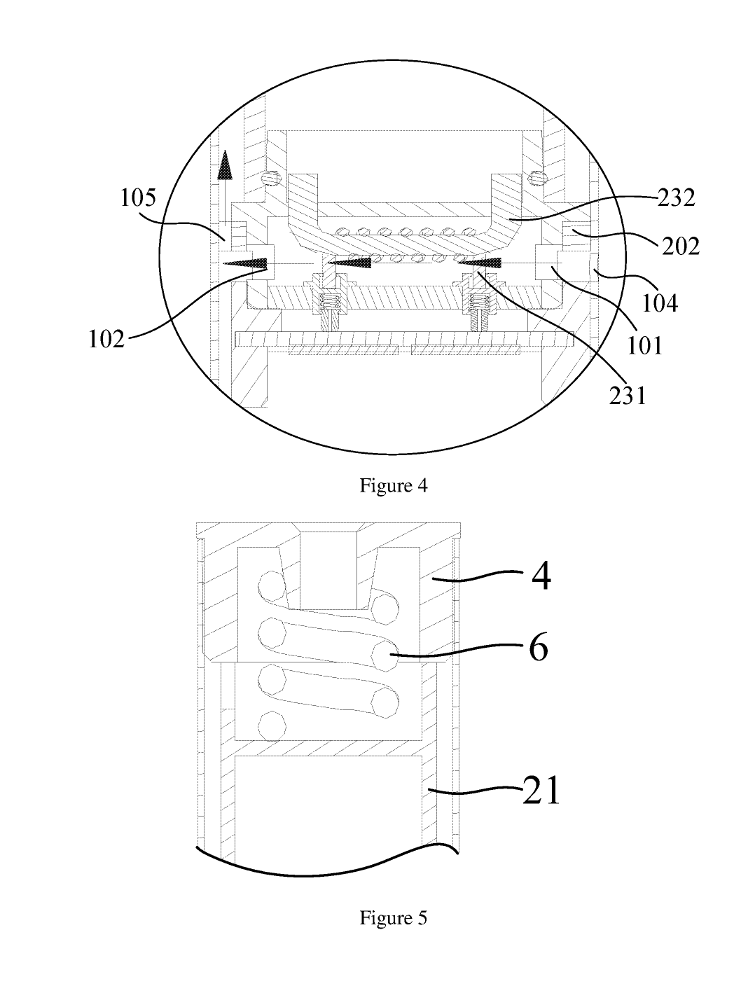

Specifically, as shown in FIG. 4, FIG. 4 is an enlarged view of the area B in FIG. 3, the disposable electronic cigarette comprises an air inlet hole 101, an air outlet hole 102, an air intake port 104, a ventilation groove 105, a first sealing seat 202, an electric heating wire 231 and an e-liquid guiding wick 232. The air inlet hole 101 and the air outlet hole 102 are defined on a side wall of the atomizing chamber facing the sleeve 1, the air inlet hole 101 and the air outlet hole 102 are opposite to each other, the air inlet hole 101 is communicated with the outside of the electronic cigarette, the air outlet hole 102 is communicated with the smoke passage. Understandably, as the air inlet hole 101 and the air outlet hole 102 are defined on a side wall of the atomizing chamber, the air inlet hole 101 and the air outlet hole 102 which are opposite to each other may be defined at a circumferential sidewall of an end of the cup cover 22 contacted with an atomizing seat 24, or the air inlet hole 101 and the air outlet hole 102 may be defined on the atomizing seat 24, it is appreciate that projections extending to the cup cover 22 at a peripheral edge of the atomizing seat 24, and the air inlet hole 101 and the air outlet hole 102 opposite to each other are formed in an inner wall of the projections. In the present embodiment, the air inlet hole 101 and the air outlet hole 102 are provided on a circumferential side wall of the cup cover 22, the electronic cigarette further comprises an air intake passage, the air intake passage, the atomizing chamber and the smoke passage form an airflow passage together.

In the present embodiment, a formation of the air intake passage is as follows: the sleeve 1 is provided with an the air intake port 104 corresponding to the air inlet hole 101, outside air enters into the atomizing chamber by sequentially passing through the air intake port 104 and the air inlet hole 101 so as to form the air intake passage. In order to allow smoke formed in the atomizing chamber to flow only in the direction opposite to the air intake port 104 to finally flow into the user's mouth, a first sealing seat 202 is defined between the cup cover 22 and the sleeve 1, one side of the first sealing seat 202 close to the air outlet hole 102 is provided with a ventilation groove 105 for communicating the atomizing chamber with the smoke passage, smoke in the atomizing chamber flows sequentially from the air outlet hole 102 and the ventilation groove 105 into the smoke passage, so as to avoid that atomized smoke stays in the atomizing chamber and affects a taste of the smoke and users experience. Thus to realized that the airflow enters from one side of the sleeve 1 and carries the smoke in the atomizing chamber to be discharged from the other side of the sleeve 1, so as to avoid the airflow channel bending too many times to result in phenomenon of airflow noise and suffocation.

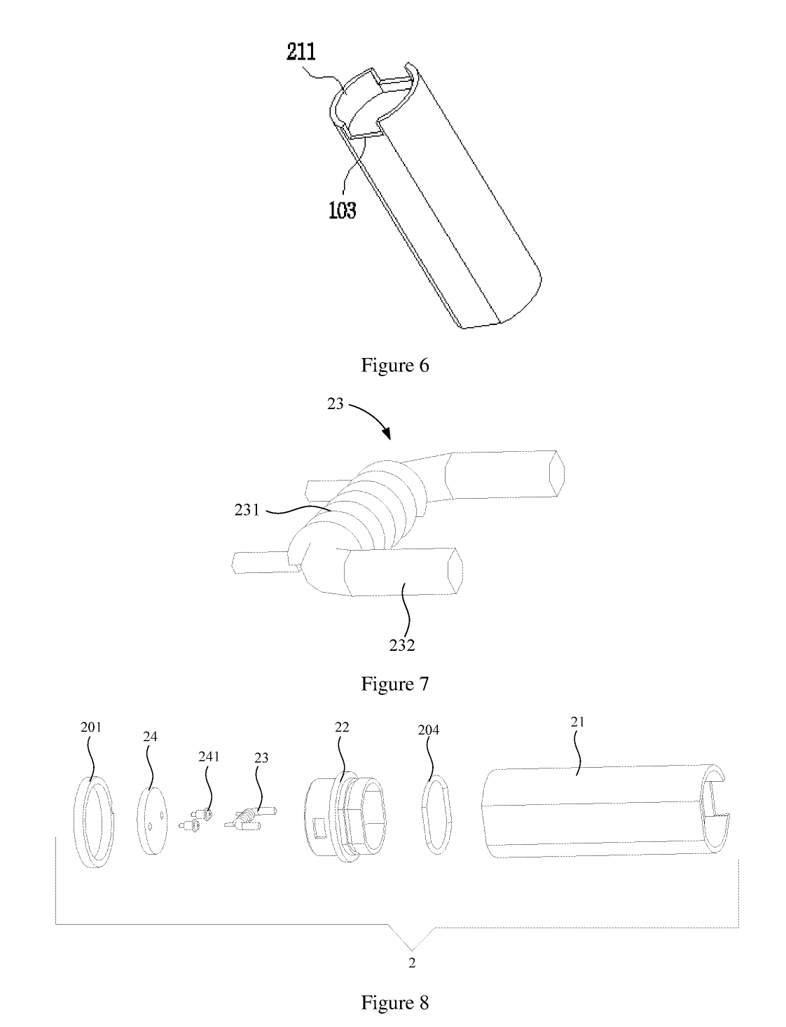

As shown in FIG. 6, a circumference of one end of the e-liquid cup 21 opposite to the cup cover 22 extends along an end opposite to the cup cover 22 to form an abutting sleeve 211, a smoke hole 103 corresponding to the air outlet hole 102 is provided in a circumferential side wall of the abutting sleeve 211, the smoke is discharged from the smoke hole 103.

In the present embodiment, as shown in FIG. 7, a heating wire assembly 23 comprises the electric heating wire 231 and the e-liquid guiding wick 232 for winding the electric heating wire 231 and for guiding e-liquid, different with the electric heating wire in a spiral disk shape of the prior art, a contact area between the electric heating wire 231 and the e-liquid is small to prevent the contact area between the electric heating wire and the e-liquid is too large and lose the heat fast resulting in phenomenon that the e-liquid cannot be atomized.

As shown in FIG. 8, FIG. 8 is an explosion diagram of the atomizing assembly of the first embodiment of the disposable electronic cigarette of the present invention, in the first embodiment of the present invention, the atomizing assembly 2 comprises the e-liquid cup 21, the cup cover 22, the heating wire assembly 23, conductive devices 241 and the atomizing seat 24. A sealing ring 204 is defined between the cup cover 22 and the e-liquid cup 21, the sealing ring 204 is configured for sealing the e-liquid, and preventing phenomenon of e-liquid leaking when the cup cover 22 is inserted into the e-liquid cup 21. An elastic sealing device 201 is defined between the atomizing assembly 2 and the battery component 3 to prevent atomized smoke from entering the battery component 3 to affect a normal operation of the battery. The cup cover 22 is provided on an opening of the e-liquid cup 21, and the atomizing seat 24 and the cup cover 22 enclose to form the atomizing chamber, namely, one end of the cup cover 22 and the atomizing seat 24 in a plate shape enclose to form the atomizing chamber, one end of the heating wire assembly 23 is connected to the conductive devices 241, the heating wire assembly 23 and the conductive devices 241 are placed in the atomizing chamber, and one ends of the conductive devices 241 opposite to the heating wire assembly 23 are inserted into the atomizing seat 24.

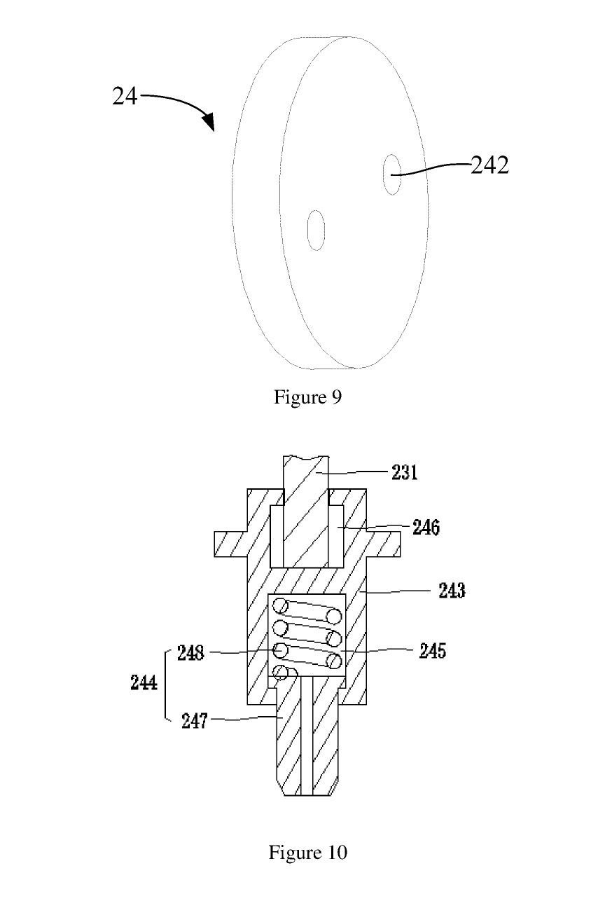

Specifically, as shown in FIG. 9, two through holes 242 spaced from each other are axially defined along on the atomizing seat 24, each of the through holes 242 is provided with each of the conductive devices 241 (shown as FIG. 8).

Further, as shown in FIG. 10, each of the conductive devices 241 comprises a fixing sleeve 243 and an elastic column 244, a first axial mounting position 245 and a second axial mounting position 246 are respectively defined in the fixing sleeve 243, the elastic column 244 is elastically and movably mounted in the first axial mounting position 245, each end of two ends of the electric heating wire 231 is respectively and correspondingly inserted into the second axial mounting position 246. Specifically, the elastic column 244 comprises an abutment member 247 movably defined in the first axial mounting position 245 and configured for being electrically connected to the battery component 3 (shown as FIG. 5); and an elastic member 248 provided in the first axial mounting position 245 for providing an elastic force for the abutment member 247 and resetting the abutment member 247, when the battery component 3 is connected to the atomizing assembly 2, the battery 32 in the battery component 3 is electrically connected with the abutment member 247. The elastic member 248 may be a spring or a rubber or the like, and the elastic reset of the elastic member 248 may depend on a retraction distance of the spring or the rubber. A outer diameter of the fixing sleeve 243 is adapted to the through holes 242 (shown in FIG. 9), the fixing sleeve 243 is fixed in one through hole 242, an opening diameter of the second axial mounting position 246 corresponds to the electric heating wire 231 to ensure that the electric heating wire 231 is stably accommodated in the second axial mounting position 246. In the present embodiment, the elastic member 248 is a spring, and the spring is configured as the force applying means at the same time.

As shown in FIG. 11, the cup cover 22 comprises an e-liquid separator 221, a first connecting portion 224 connected to one end of the e-liquid separator 221 and inserted into the e-liquid cup 21, a second connecting portion 225 connected to the other end of the e-liquid separator 221, the air inlet hole 101 and the air outlet hole 102 are oppositely provided in a circumferential side wall of the second connecting portion 225, and an outer peripheral wall of the e-liquid separator 221 is provided with a placing groove 223 for placing a sealing ring 204, the placing groove 223 is an annular groove formed on the outer peripheral wall of the e-liquid separator 221, and when assembled, an annular seal ring 204 is placed on the placing groove 223 and then inserted into the opening of the e-liquid cup 21 together with the cup cover 22.

In the present embodiment, the cup cover 22 is provided with the e-liquid separator 221 for blocking a leakage of e-liquid, the e-liquid permeating holes 222 are defined on the e-liquid separator 221, two ends of the e-liquid guiding wick 232 (shown in FIG. 5) are respectively inserted into the e-liquid permeating holes 222 and extend to the e-liquid cup 21 to directly contact with the e-liquid. In the present embodiment, the e-liquid is directly guided out through the e-liquid guiding wick 232.

The atomizing seat 24 is inserted into the second connecting portion 225, the second connecting portion 225 and the atomizing seat 24 enclose to form the atomizing chamber (as shown in FIG. 4); the second connecting portion 225 and the atomizing seat 24 are abutted against the elastic sealing device 201, and a gap is defined between an outer side wall of the e-liquid separator 221 and the sleeve 1 for flowing the smoke.

### A Second Embodiment

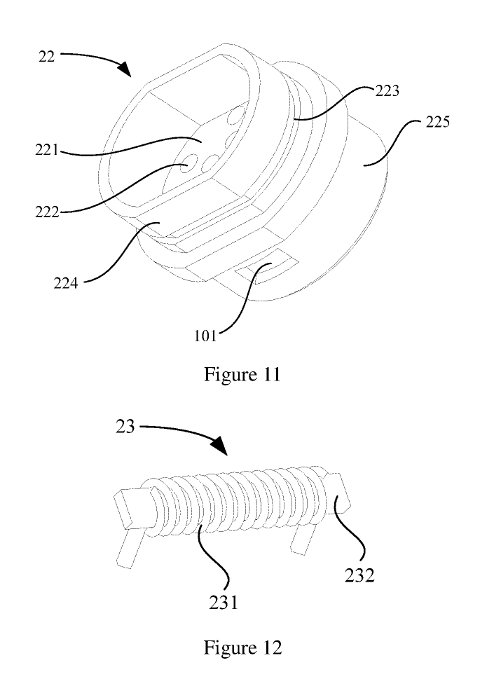

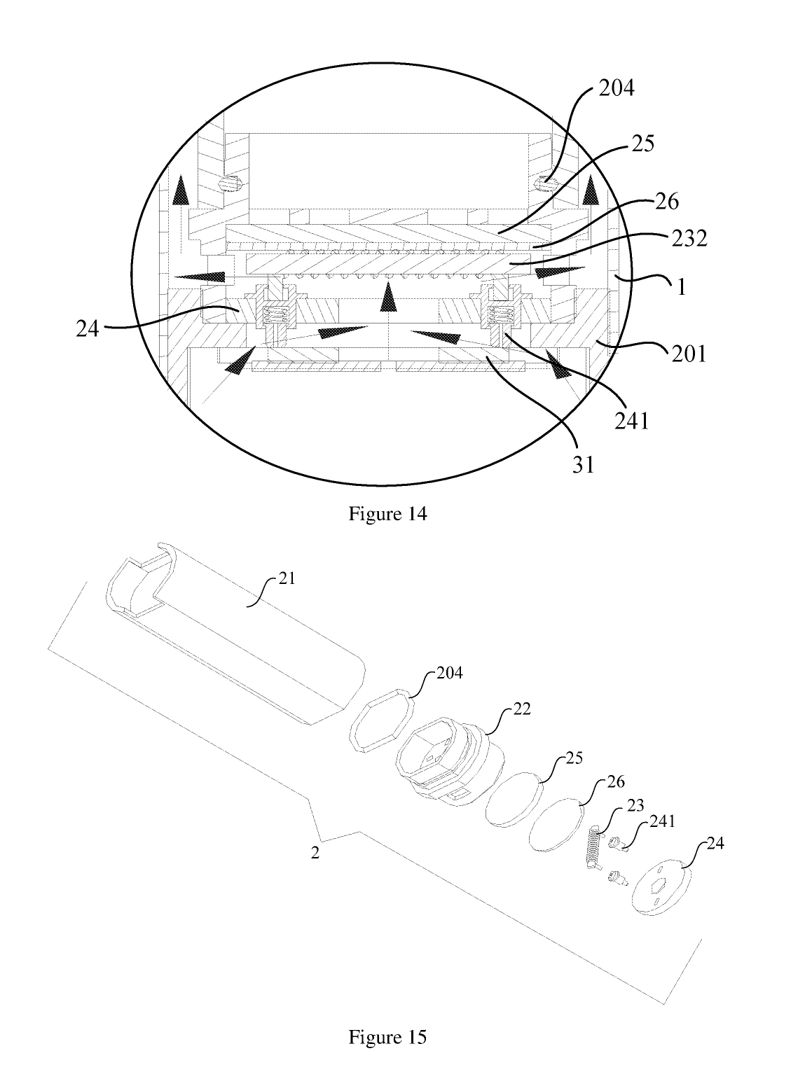

As shown in FIG. 12, in the second embodiment of the present invention, the second embodiment differs from the first embodiment in the structure of the electric heating wire 231 and an addition of the e-liquid storage cotton 25 and the fiberglass sheet 26 in the atomizing chamber (as shown in FIG. 14). The heating wire assembly 23 comprises an electric heating wire 231 and an e-liquid guiding wick 232 for winding the electric heating wire 231 and for guiding e-liquid. As shown in FIG. 11, an e-liquid separator 221 for blocking a leakage of e-liquid is provided in a cup cover 22, e-liquid permeating holes 222 are defined on the e-liquid separator 221. In the present embodiment, the e-liquid permeating holes 222 are different from those of the first embodiment, and the e-liquid permeating holes 222 of the present embodiment is a plurality of through holes provided on the e-liquid separator 221, and the e-liquid leaks directly from the e-liquid permeating holes 222, an e-liquid storage cotton 25 is abutted against an end surface of the e-liquid separator 221 opposite to e-liquid, the e-liquid storage cotton 25 guides the e-liquid out from the e-liquid permeating holes 222; a fiberglass sheet 26 is abutted against an end surface of e-liquid storage cotton 25, the end surface of e-liquid storage cotton 25 is opposite to the e-liquid separator 221, a part of an end surface of the electric heating wire 231 wound on the e-liquid guiding wick 232 is abutted against the fiberglass sheet 26, In the present embodiment, the e-liquid is transferred to the e-liquid storage cotton 25 through the e-liquid permeating holes 222 and flows from the e-liquid storage cotton 25 to the fiberglass sheet 26 and then comes into contact with the electric heating wire 231 to prevent the heat generated by the high temperature heating of the heating wire assembly 23 producing cotton burning phenomenon.

### A Third Embodiment

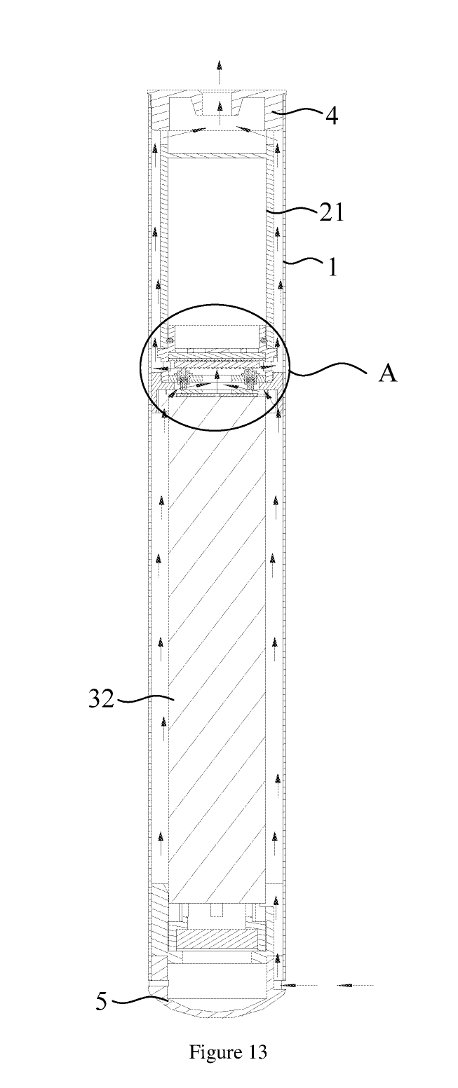

FIG. 13 is a front view of a third embodiment of the disposable electronic cigarette of the present invention, the disposable electronic cigarette comprises a sleeve 1, a suction nozzle cover 4, an end cap 5, and an e-liquid cup 21 and a battery 32, the suction nozzle cover 4 and the end cap 5 are provided at two ends of the sleeve 1, and the e-liquid cup 21 and the battery 32 are co-axially defined in the sleeve 1, and the e-liquid cup 21 is defined at one end of the sleeve 1 close to the suction nozzle cover 4, and the battery 32 is defined at one end of the sleeve 1 close to the end cap 5. Compared with the first embodiment, airflow of the present embodiment flows from one end of the end cap 5 to the atomizing chamber and then from the atomizing chamber to one end of the suction nozzle cap 4. In the present embodiment, a battery component 3 is co-axially defined with the atomizing assembly 2, and the battery component 3 and the atomizing assembly 2 are defined within the sleeve 1, a first gap is defined between the atomizing assembly 2 and the sleeve 1, the first gap forms the smoke passage, a second gap is defined between the battery component 3 and the sleeve 1, and the second gap forms the air intake passage. Air enters the second gap from one end of the sleeve 1 close to the end cap 5 and then enters the atomizing chamber from the second gap, and finally the smoke in the atomizing chamber is removed from the first gap.

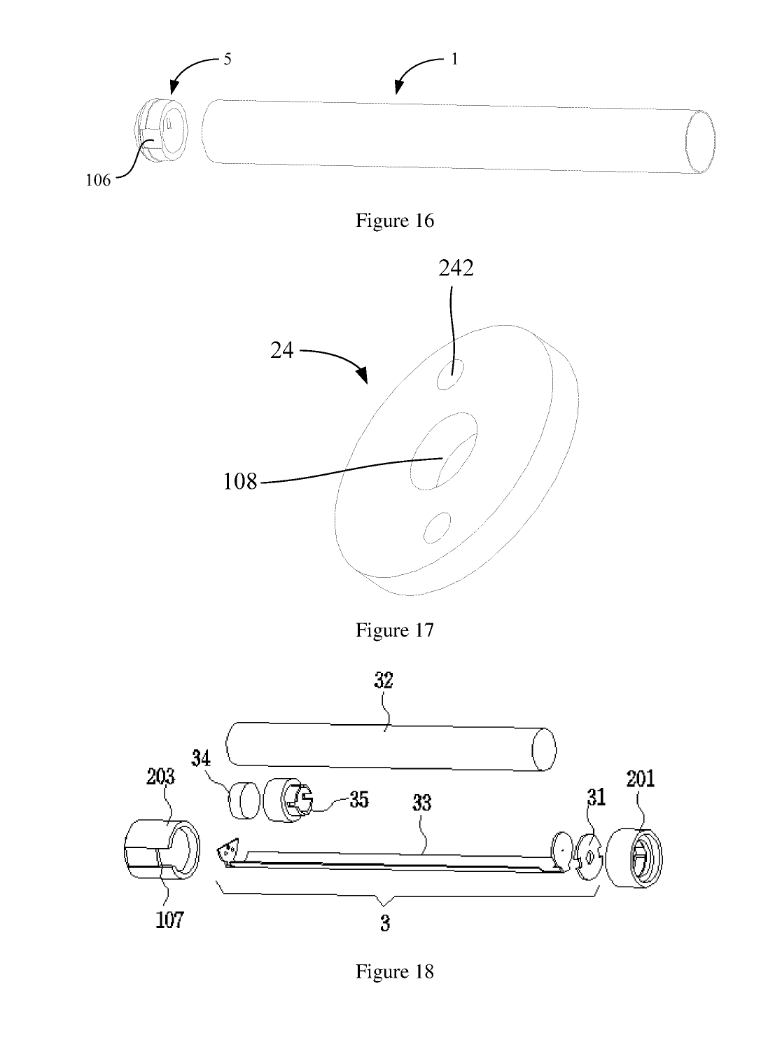

FIG. 14 is an enlarged view of the section A in FIG. 13, FIG. 15 is an explosion view of an atomizing assembly of the third embodiment of the disposable electronic cigarette of the present invention, in the present embodiment, a heating wire assembly 23 is the same as the heating wire assembly 23 in the second embodiment, It is to be understood that, in other embodiments of the present invention, the heating wire assembly 23 may be the same as the heating wire assembly 23 in the first embodiment. A first intake slot 106 is defined on the end cap 5, the first intake slot 106 is not communicated with an inner cavity of the end cap 5 (as shown in FIG. 16), a second intake slot 107 corresponding to the first intake slot 106 is defined on the second sealing seat 203 (as shown in FIG. 18), a center through hole 108 is defined on the atomizing seat 24 (as shown in FIG. 17), outside air passes through the first intake slot 106, the second intake groove 107 and the central through hole 108 into the atomizing chamber. As shown in FIG. 13, since the inner cavity of the end cap 5 communicates with the outside air and is separated from the first intake slot 106, the air pressure difference between the inner cavity of the end cap 5 and an airflow direction is significant, so that the airflow sensor is easy to sense airflow changes, that is, to improve sensitivity of the airflow sensor to solve the problem that the air pressure at two ends of the airflow sensor is small and the sensitivity is low when the airflow needs to flow through the airflow sensor in the prior art.

In the first embodiment, the second embodiment and the third embodiment of the present invention, it is understood that an opening is provided in the suction nozzle cover 4, and the smoke is discharged through the opening after passing through the smoke passage.

FIG. 18 is an explosion diagram of the battery component, the elastic sealing device and the second sealing seat of the disposable electronic cigarette of the present invention. the battery component 3 comprises a circuit board 31 abutted against the conductive devices 241, a battery 32, a FPC board 33, an airflow sensor 34 and an airflow sensor seat 35 which is configured for accommodating the airflow sensor 34, the circuit board 31 and the airflow sensor seat 35 are defined at two ends of the FPC board 33, respectively, the battery 32 is provided in the FPC board 33, the circuit board 31 is configured for realizing an electrical connection between the battery 32 and the airflow sensor seat 34, the second sealing seat 203 is sleeved on the battery 32 and the airflow sensor seat 35 and is defined at one end of the battery component 3, the elastic sealing device 201 sleeves on the circuit board 31 and sleeves on the other end of the battery component 3 to enable the battery component 3 to be inserted into one end of the sleeve 1 with the elastic sealing device 201 and the second sealing seat 203 being sleeved on two ends when assembling. When assembling, it is only necessary to insert the battery component 3 into one end of the sleeve 1, thereby facilitating an automated production and improving a production efficiency. In the present invention, the airflow sensor 34 is preferably a microphone head.

In the present invention, the circuit board 31 is preferably of the same shape as the FPC board 33 (not shown in drawings), one end of the circuit board 31 close to the airflow sensor 34 is provided with three conductive holes, each of the conductive holes is in contact with three contact points on the airflow sensor 34, the conductive holes are also in electrical contact with one end of the battery 32, the other end of the circuit board 31 is in electrical contact with the other end of the battery 32, namely, a positive electrode and a negative electrode of the battery 32 are in contact with the two ends of the circuit board 31, respectively. The battery 32, the airflow sensor 34 and the airflow sensor seat 35 are embedded in a frame chamber of the FPC board 33 and are limited by mutually pressing of limiting boards defined at two ends of the FPC board 33 to achieve an electrical connection.

In the present invention, an outer surface of the e-liquid cup 21 comprises an axially extending section and a remaining surface (not shown in the drawings), the section and an inner wall of the sleeve 1 form the smoke passage, the remaining surface is attached to the inner wall of the sleeve 1. Then a contact area of smoke and sleeve 1 is reduced to reduce smoke condensation, so that more smoke can reach into the user mouth to enhance the user experience.

Generally, applications of the disposable electronic cigarette of the present invention have following advantages, through a configuration of the e-liquid cup 21, the e-liquid cup 21 can be more conveniently inserted into the sleeve 1 directly in the assembly process of the electronic cigarette, a degree of an automated production is high, and by providing the seal ring 204 for sealing the e-liquid on the cup cover 22, it is possible to prevent the e-liquid leakage when the cup cover 22 is inserted into the e-liquid cup 21; By applying the force applying device on the conductive devices 241 in a direction to the battery component 3, the conductive devices 241 can be brought into close to contact with the battery component 3, besides, the conductive devices 241 are elastic devices, thereby eliminating phenomenon of an electrical contact failure; An elastic sealing device 201 defined between the atomizing assembly 2 and the battery component 3 prevents the smoke atomized by the atomizing assembly 2 from entering the battery component 3 to affect a normal operation of the battery; By providing the air intake port 104 on the sleeve 1, airflow is introduced into the atomizing chamber through the air intake port 104 to carry the smoke out from the other side of the e-liquid cup 21 to the user mouth, and the airflow path is prevented from being bent too many times to produce phenomenon of airflow noise and suffocation; The first sealing seat 202 is defined on the outer peripheral surface of the atomizing chamber, and a ventilation groove 105 is defined on one side of the first sealing seat 202 so that the smoke is discharged from the ventilation groove 105 to prevent the atomized smoke from accumulating in the atomizing chamber to affect smoke taste and user experience; Through a small abutting area between surfaces of the electric heating wire 231 and the fiberglass sheet 26 to ensure a smooth e-liquid transmission, but also to prevent that the contact area between the electric heating wire 231 and the e-liquid is too large and lose the heat fast resulting in phenomenon that the e-liquid cannot be atomized; By directing two ends of the e-liquid guiding wick 232 into the e-liquid cup 21 and the direct contact with the e-liquid, it is possible to avoid the phenomenon that the e-liquid provided for the electric heating wire 231 to atomize is insufficient to produce burning cotton phenomenon; By embedding the airflow sensor seat 35 and the airflow sensor 34 into the second sealing seat 203, it is possible to prevent the airflow sensor 34 from receiving no triggered and sensed signal due to a small air pressure difference inside and outside airflow sensor 34 then the electronic cigarette cannot work normally.

While the embodiments of the present application are described with reference to the accompanying drawings above, the present application is not limited to the above-mentioned specific implementations. In fact, the above-mentioned specific implementations are intended to be exemplary not to be limiting. In the inspiration of the present application, those ordinary skills in the art can also make many modifications without breaking away from the subject of the present application and the protection scope of the claims. All these modifications belong to the protection of the present application.

1. A disposable electronic cigarette comprising a sleeve (1), an atomizing assembly (2) and a battery component (3) which are sleeved within the sleeve (1), wherein the atomizing assembly (2) comprises an e-liquid cup (21) provided with an opening at an end of the e-liquid cup (21) and configured for storing an e-liquid, the end of the e-liquid cup (21) faces the battery component (3); at least one smoke passage is defined between the sleeve (1) and the e-liquid cup (21) for discharging atomized smoke outside one end of the atomizing assembly (2); a cup cover (22) is connected to the e-liquid cup (21) and covers the opening of the e-liquid cup (21), the cup cover (22) is provided with e-liquid permeating holes (222) for guiding the e-liquid out of the e-liquid cup (21); a heating wire assembly (23) is provided for atomizing the e-liquid outputted from the e-liquid permeating holes (222); an atomizing seat (24) is provided for fixing the heating wire assembly (23), an atomizing chamber for receiving the heating wire assembly (23) is enclosed between the atomizing seat (24) and the cup cover (22), the atomizing chamber is communicated with the smoke passage;
the electronic cigarette further comprises conductive devices (241) and a force applying device, the conductive devices (241) are defined axially along the atomizing seat (24), the force applying device is configured for applying a force on the conductive devices (241) in a direction to the battery component (3), one ends of the conductive devices (241) are connected to the heating wire assembly (23), and the other ends of the conductive devices (241) are elastically abutted against the battery component (3) by a force of the force applying device; an elastic sealing device (201) for preventing the e-liquid in the atomizing assembly (2) from leaking toward the battery component (3) is defined between the atomizing assembly (2) and the battery component (3), the elastic sealing device (201) is elastically abutted against an inner wall of the sleeve (1).

2. The disposable electronic cigarette according to claim 1, wherein an air inlet hole (101) and an air outlet hole (102) are defined on a side wall of the atomizing chamber, the side wall of the atomizing chamber faces the sleeve (1), the air inlet hole (101) and the air outlet hole (102) are opposite to each other, the air inlet hole (101) is communicated with the outside of the electronic cigarette, the air outlet hole (102) is communicated with the smoke passage.

3. The disposable electronic cigarette according to claim 2, wherein an e-liquid collection tank for collecting leaked and condensed e-liquid is formed in one end of the atomizing chamber, the end of the atomizing chamber is opposite to the e-liquid cup (21).

4. The disposable electronic cigarette according to claim 3, wherein the air inlet hole (101) and the air outlet hole (102) are provided on a circumferential side wall of the cup cover (22), a circumference of one end of the e-liquid cup (21) extends along a direction opposite to the cup cover (22) to form an abutting sleeve (211), the one end of the e-liquid cup (21) is opposite to the cup cover (22), a smoke hole (103) corresponding to the air outlet hole (102) is provided in a circumferential side wall of the abutting sleeve (211);
the electronic cigarette further comprises an air intake passage, the air intake passage, the atomizing chamber and the smoke passage form an airflow passage together.

5. The disposable electronic cigarette according to claim 4, wherein the sleeve (1) is provided with an air intake port (104) corresponding to the air inlet hole (101), outside air enters into the atomizing chamber by sequentially passing through the air intake port (104) and the air inlet hole (101), so as to form the air intake passage.

6. The disposable electronic cigarette according to claim 5, wherein a first sealing seat (202) is defined between the cup cover (22) and the sleeve (1), one side of the first sealing seat (202) close to the air outlet hole (102) is provided with a ventilation groove (105) for communicating the atomizing chamber with the smoke passage, smoke flows from the atomizing chamber into the smoke passage through the ventilation groove (105) and is discharged from the smoke passage.

7. The disposable electronic cigarette according to claim 1, wherein the heating wire assembly (23) comprises an electric heating wire (231) and an e-liquid guiding wick (232), the e-liquid guiding wick (232) is configured for guiding e-liquid, the electric heating wire (231) is wound around the e-liquid guiding wick (232), two through holes (242) spaced from each other are axially defined in the atomizing seat (24), each of the through holes (242) is provided with one of the conductive devices (241), each of the conductive devices (241) comprises a fixing sleeve (243) and an elastic column (244), a first axial mounting position (245) and a second axial mounting position (246) are respectively and correspondingly defined in the fixing sleeve (243), the elastic column (244) is elastically and movably mounted in the first axial mounting position (245), two ends of the electric heating wire (231) are respectively and correspondingly inserted into two second axial mounting positions (246) of the conductive devices (241).

8. The disposable electronic cigarette according to claim 7, wherein the elastic column (244) comprises an abutment member (247) movably defined in the first axial mounting position (245) and configured for being electrically connected to the battery component (3), and an elastic member (248) provided in the first axial mounting position (245) for providing an elastic force for the abutment member (247) and resetting the abutment member (247), when the battery component (3) is connected to the atomizing assembly (2), the battery (32) in the battery component (3) is electrically connected with the abutment member (247).

9. The disposable electronic cigarette according to claim 8, wherein an e-liquid separator (221) for blocking a leakage of e-liquid is provided in the cup cover (22), the e-liquid permeating holes (222) are defined on the e-liquid separator (221), an e-liquid storage cotton (25) is abutted against an end surface of the e-liquid separator (221), the end surface of the e-liquid separator (221) is opposite to e-liquid, the e-liquid storage cotton (25) guides the e-liquid out from the e-liquid permeating holes (222); a fiberglass sheet (26) is abutted against an end surface of e-liquid storage cotton (25), the end surface of e-liquid storage cotton (25) is opposite to the e-liquid separator (221), the electric heating wire (231) is wound around the e-liquid guiding wick (232), the e-liquid guiding wick (232) is abutted against the fiberglass sheet (26).

10. The disposable electronic cigarette according to claim 8, wherein an e-liquid separator (221) for blocking a leakage of e-liquid is provided in the cup cover (22), the e-liquid permeating holes (222) are defined on the e-liquid separator (221), two ends of the e-liquid guiding wick (232) are respectively inserted into the e-liquid permeating holes (222) and extend to the e-liquid cup (21) to directly contact with the e-liquid.

11. The disposable electronic cigarette according to claim 1, wherein the electronic cigarette further comprises an end cap (5) inserted into one end of the sleeve (1), the end of the sleeve (1) is close to the battery component (3), and a second sealing seat (203) is defined between the battery component (3) and the end cap (5);
a first intake slot (106) is defined on the end cap (5), a second intake slot (107) corresponding to the first intake slot (106) is defined on the second sealing seat (203), a center through hole (108) is defined on the atomizing seat (24), outside air successively passes through the first intake slot (106), the second intake slot (107) and the center through hole (108) to enter the atomizing chamber.

12. The disposable electronic cigarette according to claim 11, wherein the battery component (3) is co-axially defined with the atomizing assembly (2), and the battery component (3) and the atomizing assembly (2) are defined within the sleeve (1), a first gap is defined between the atomizing assembly (2) and the sleeve (1), the first gap forms the smoke passage, a second gap is defined between the battery component (3) and the sleeve (1), and the second gap forms an air intake passage.

13. The disposable electronic cigarette according to claim 12, wherein the electronic cigarette further comprises a suction nozzle cover (4) inserted into one end of the sleeve (1) close to the e-liquid cup (21) and abutted against the e-liquid cup (21), the suction nozzle cover (4) is provided with an opening, smoke is discharged through the opening after passing through the smoke passage.

14. The disposable electronic cigarette according to claim 1, wherein the battery component (3) comprises a circuit board (31) abutted against the conductive devices (241), a battery (32), a FPC board (33), an airflow sensor (34) and an airflow sensor seat (35), the airflow sensor seat (35) is configured for accommodating the airflow sensor (34), the circuit board (31) and the airflow sensor seat (35) are defined at two ends of the FPC board (33), respectively, the battery (32) is provided on a side of the FPC board (33) and is located between the circuit board (31) and the airflow sensor seat (35), the FPC board (33) is configured for realizing an electrical connection between the circuit board (31) and the airflow sensor (34);
a second sealing seat (203) is sleeved on the battery (32) and the airflow sensor seat (35) and is defined at one end of the battery component (3), the elastic sealing device (201) sleeves on the circuit board (31) and sleeves on the other end of the battery component (3) to enable the battery component (3) to be inserted into one end of the sleeve (1) with the elastic sealing device (201) and the second sealing seat (203) being sleeved on two ends of the battery component (3) when assembling.

15. The disposable electronic cigarette according to claim 14, wherein one end of the FPC board (33) is provided with three conductive holes, the end of the FPC board (33) is close to the airflow sensor (34), one of the conductive holes is in contact with one of three contact points on the airflow sensor (34), the conductive holes are also in electrical contact with one end of the battery (32), the other end of the FPC board (33) is in electrical contact with the other end of the battery (32), the battery (32), the airflow sensor (34) and the airflow sensor seat (35) are embedded in a frame chamber of the FPC board (33) and are limited by mutually pressing of limiting boards defined at two ends of the FPC board (33) to achieve an electrical connection.

16. The disposable electronic cigarette according to claim 1, wherein the cup cover (22) comprises an e-liquid separator (221), a first connecting portion (224) connected to one end of the e-liquid separator (221) and inserted into the e-liquid cup (21), a second connecting portion (225) connected to the other end of the e-liquid separator (221), an air inlet hole (101) and an air outlet hole (102) are oppositely provided in a circumferential side wall of the second connecting portion (225), and an outer peripheral wall of the e-liquid separator (221) is provided with a placing groove (223) for placing a sealing ring (204).

17. The disposable electronic cigarette according to claim 16, wherein the atomizing seat (24) is inserted into the second connecting portion (225), the atomizing seat (24) and the second connecting portion (225) enclose to form the atomizing chamber; the second connecting portion (225) and the atomizing seat (24) are abutted against the elastic sealing device (201), and a gap for smoke to flow through is provided between an outer side wall of the e-liquid separator (221) and the sleeve (1).

18. The disposable electronic cigarette according to claim 1, wherein an outer surface of the e-liquid cup (21) comprises an axially extending section and a remaining surface, the section and an inner wall of the sleeve (1) form the smoke passage, the remaining surface is attached to the inner wall of the sleeve (1).

19. The disposable electronic cigarette according to claim 1, wherein the force applying device comprises a first magnet and a second magnet, the first magnet and the second magnet are defined on the atomizing seat (24) and the battery component (3), respectively.

20. The disposable electronic cigarette according to claim 1, wherein the electronic cigarette further comprises a suction nozzle cover (4) inserted into the one end of sleeve (1), the one end of sleeve (1) is close to the e-liquid cup (21); the force applying device is a spring (6), two ends of the spring (6) are abutted against an end of the suction nozzle cover (4) and an end of the e-liquid cup (21), respectively.
